(19) United States
(12) Patent Application Publication
(10) Pub. No.: US 2009/0255538 A1
(43) Pub. Date: Oct. 15, 2009

(54) Monolithic endotracheal tube holder

(75) Inventors: Glen Thomson, Citrus Heights, CA (US); John D. Moltzner, Elk Grove, CA (US)

Correspondence Address: James Lee;VORTRAN Medical Technology 1, Inc., 21 Golden Land Court, Sacramento, CA, 95834, US

(73) Assignee: VORTRAN Medical Technology 1, Inc., Sacramento, CA (US)

(21) Appl. No.: 12/384,921

(22) Filed: Apr. 10, 2009

Related U.S. Application Data
(60) Provisional application No. 61/124,487, filed on Apr. 14, 2008.

(51) Int. Cl. A61M 16/04

(52) U.S. Cl. 128/207.17

(57) ABSTRACT

This invention pertains generally to a device for retaining a medically relevant tube into a proper registration for application of medical treatment to a patient, and more particularly to a flexible holder for positioning of an endotracheal tube wherein said holder has integrated therein circumferential guard projections for maintaining optimal flow characteristics of the restrained endotracheal tube. Circumferential guard projections extend both outwardly and inwardly from said endotracheal tube holder and are integral to and monolithically formed with the endotracheal tube holder. The endotracheal tube holder is adaptable to receiving tubes of varying diameters and includes a capture means for allowing insertion and removal of an endotracheal tube through a transverse access port in a side aspect of the holder. The monolithic nature of the endotracheal tube holder design is further enhanced through incorporation of access portals about the holder for allowing routine patient maintenance.

This application claims the benefit under 35 U.S.C. 119(e) of U.S. provisional applications Ser. No. 61/124,487 filed Apr. 14, 2008, which is incorporated by reference herein in its entirety.

### BACKGROUND OF THE INVENTION

An endotracheal tube is typically employed in acute respiratory support, such as in operative anesthesia, intensive care, and emergency medicine for airway management and mechanical ventilation. The endotracheal tube is inserted into and through a patient's trachea in order to ensure that the airway is maintained in an open condition and that air is cycling to and from the lungs through subsequent artificial respiration. Since its inception, the endotracheal tube has been regarded by the medical profession as the most reliable available method for protecting a patient's airway and is routinely taught as a means for establishing and maintaining patient airway integrity.

Endotracheal tubes are comprised of a primary polymeric lumen through which ventilation of the patient occurs. In order to retain the endotracheal tube in proper depth insertion through the patient trachea, an inflatable cuff about the distal or inserted end of the endotracheal tube is distended or collapsed through the action of a valved inflator associated with a secondary lumen coaxial to the primary ventilation lumen. While a small amount of force is created by the distention of the inflatable cuff upon the patient trachea, this minimal force in combination with the lubricous nature of the internal mucosa in insufficient to maintain a fixed position. The endotracheal tube is susceptible to displacement from the patient airway by incidental external forces and movement of and around the patient, with displacement of the endotracheal tube resulting in loss of the ventilation support and potentially catastrophic results.

To combat unintentional displacement of the endotracheal tube, medical personnel will at a minimum utilize adhesive tapes to restrain the endotracheal tube in an intended position and orientation for optimal patient benefit. As the application of adhesive tapes is ad hoc in nature and subject to variations inherent to different medical personnel training and experience, the effectiveness of adhesive tape as an endotracheal tube restraining means is variable and suspect. Further, adhesive tapes may be contraindicated as the chemical nature of the adhesives used in medical related tapes are found to be poorly tolerated by patient dermal contact, especially for protracted use.

The prior art presents a number of alternative means for maintaining endotracheal tube position and orientation which is reproducible in nature and overcomes the deficiencies evident in adhesive tape use. U.S. Pat. No. 7,063,088 to Christopher teaches a tube holder having an interior grip enhancer and a suspended mounting system. U.S. Pat. No. 5,345,931 to Battaglia depicts a mounting track and ET holder with cam-action lock that engages a bow section to maintain positioning. U.S. Pat. No. 4,774,944 to Mischinski involves a split block wherein the endotracheal tube is slid between the side of the split block and a locking bar is engaged. While each of these devices offer improved performance in maintaining proper orientation and placement of an endotracheal tube, due to complex design and unaddressed functional attributes, a single design has not yet been shown to be optimal.

Therefore, a need exists for an endotracheal tube holder that offer ease of rapid use, simple robust design, enhanced retention of an inserted endotracheal tube and protection of the flow characteristics of the endotracheal tube while simultaneously offering enhanced patient care.

### BRIEF SUMMARY OF THE INVENTION

This invention pertains generally to a device for retaining a medically relevant tube into a proper registration for application of medical treatment to a patient, and more particularly to a flexible holder for positioning of an endotracheal tube wherein said holder has integrated therein circumferential guard projections for maintaining optimal flow characteristics of the restrained endotracheal tube. Circumferential guard projections extend both outwardly and inwardly from said endotracheal tube holder and are integral to and monolithically formed with the endotracheal tube holder. A first circumferential guard projection extending outward from the endotracheal tube holder occupies an extraoral space from the patient and provides protection against the restrained tube from loosing flow characteristics due to external compression or avulsion forces. A second circumferential guard projection extending inward from the endotracheal tube holder occupies an intraoral space within the oral cavity of the patient and provides protection against the restrained tube from loosing flow characteristics due to compression forces caused by the patient (i.e. clenching of the mandible).

The endotracheal tube holder is adaptable to receiving tubes of varying diameters and includes a capture means for allowing insertion and removal of an endotracheal tube through a transverse access port in a side aspect of the holder. The adaptability of the present endotracheal tube holder design allows the device to be used in a broad cross section of patient sizes and ages. A traverse access port allows the holder to be opened to receive an endotracheal tube through the side thereof, thus precluding the need to disturb an established patient airway to attach or remove the endotracheal tube holder.

The endotracheal tube holder includes a base flange that extends perpendicular to the access port/circumferential guard elements and is engaged by extensions from a head band or like device so as to maintain the endotracheal tube holder in position on the patient. The base flange is an advantageous element of the monolithic design of the endotracheal tube holder as it allows for positioning of the endotracheal tube itself from a stabilized platform without reliance on the integrity of the maxilla-mandibular infrastructure for support, an infrastructure that may be severally compromised in accident victims requiring the kind of emergency respiratory care indicative of intubation and endotracheal tube use.

The monolithic nature of the endotracheal tube holder design is further enhanced through incorporation of one or more access portals in the base flange for allowing routine patient maintenance. During a course of therapy whereby an endotracheal tube is required, the ability to easily access the intraoral, as well as the pharyngeal and laryngeal regions, is extremely beneficial. It may become necessary to evacuate fluids that accumulate within the regions above the trachea. The incorporation of access portals in the base flange allow for medical personnel to insert vacuum suction devices to remove accumulated fluids and to apply medicants, particularly when protracted used of an endotracheal tube is required and maintenance of the oral tissues is necessary.

Other features and advantages of the present invention will become readily apparent from the following detailed description, the accompanying drawings, and the appended claims.

### BRIEF DESCRIPTION OF THE SEVERAL VIEWS OF THE DRAWING(S)

The invention will be more easily understood by a detailed explanation of the invention including drawings. Accordingly, drawings which are particularly suited for explaining the inventions are attached herewith; however, it should be understood that such drawings and figures are for descriptive purposes only and as thus are not necessarily to scale beyond the measurements provided. The drawings are briefly described as follows:

FIG. 1 is perspective view of an endotracheal tube holder with retention band and ring strip in accordance with the present invention.

FIG. 2 is perspective view of an endotracheal tube holder assembly.

FIG. 3 is a front view of an endotracheal tube holder assembly.

FIG. 4 is a back view of an endotracheal tube holder assembly.

FIG. 5 is a left side view of an endotracheal tube holder assembly.

FIG. 6 is a right side view of an endotracheal tube holder assembly.

FIG. 7 is a top-down view of an endotracheal tube holder assembly.

FIG. 8 is a bottom-up view of an endotracheal tube holder assembly.

FIG. 9 is a top-down view of a representative retention band.

FIG. 10 is a front view of a representative ring strip.

### DETAILED DESCRIPTION OF THE INVENTION

While the present invention is susceptible of embodiment in various forms, there is shown in the drawings and will hereinafter be described a presently preferred embodiment of the invention, with the understanding that the present disclosure is to be considered as an exemplification of the invention, and is not intended to limit the invention to the specific embodiment illustrated.

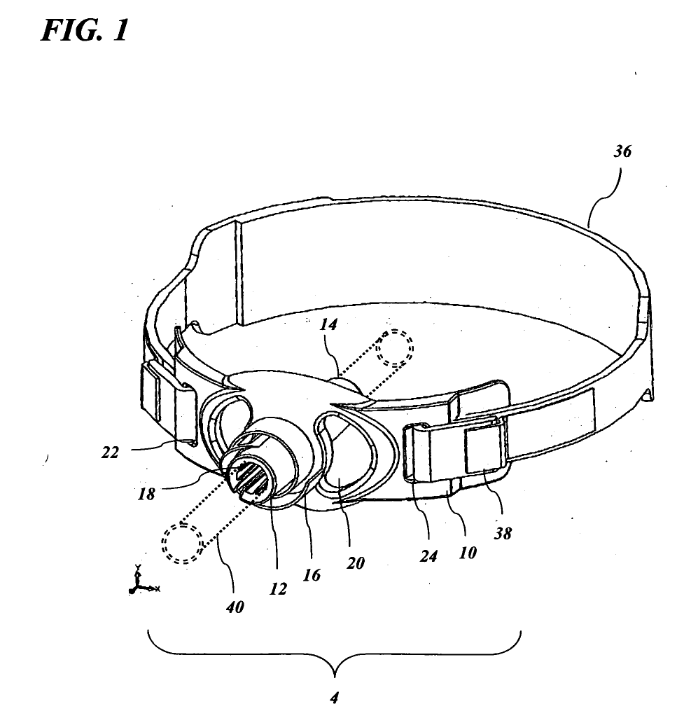

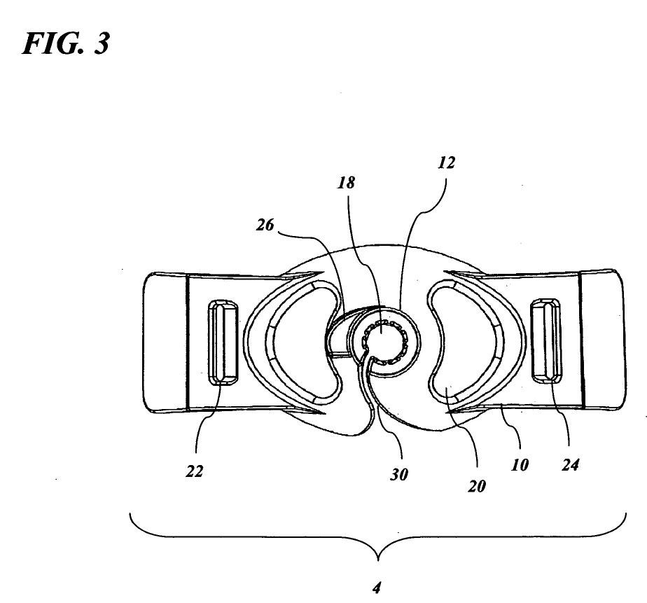

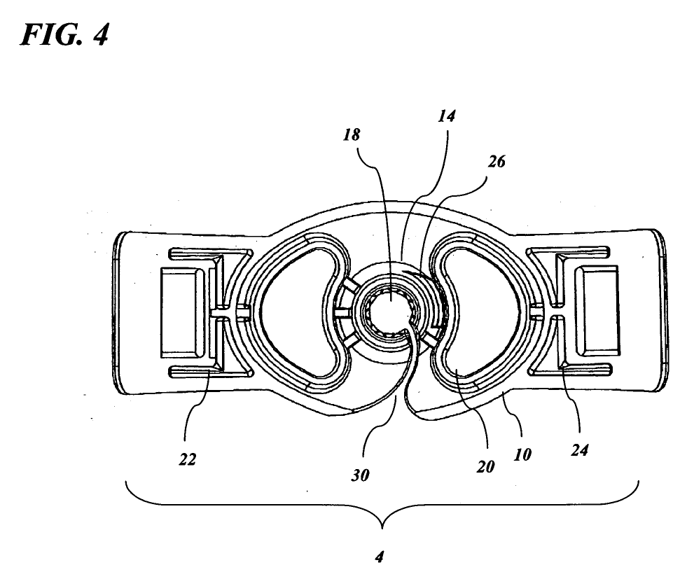

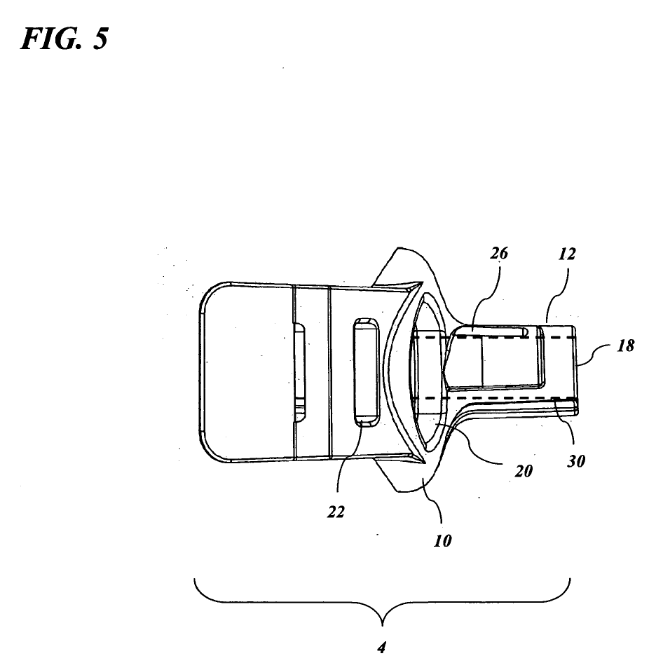

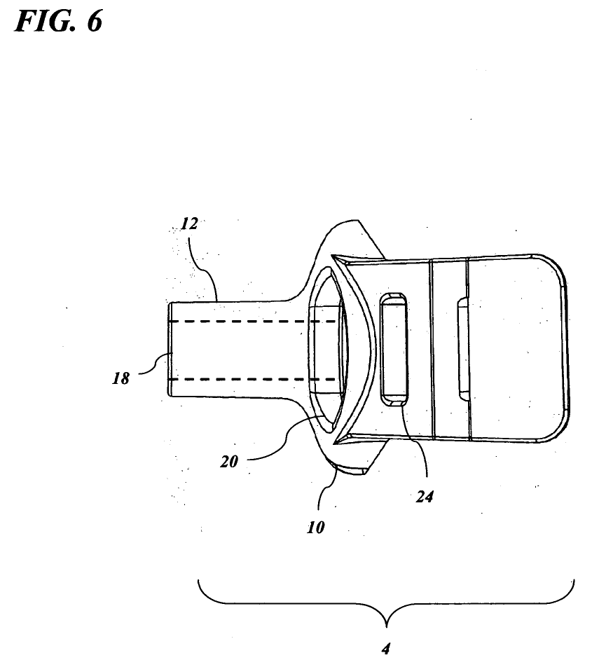

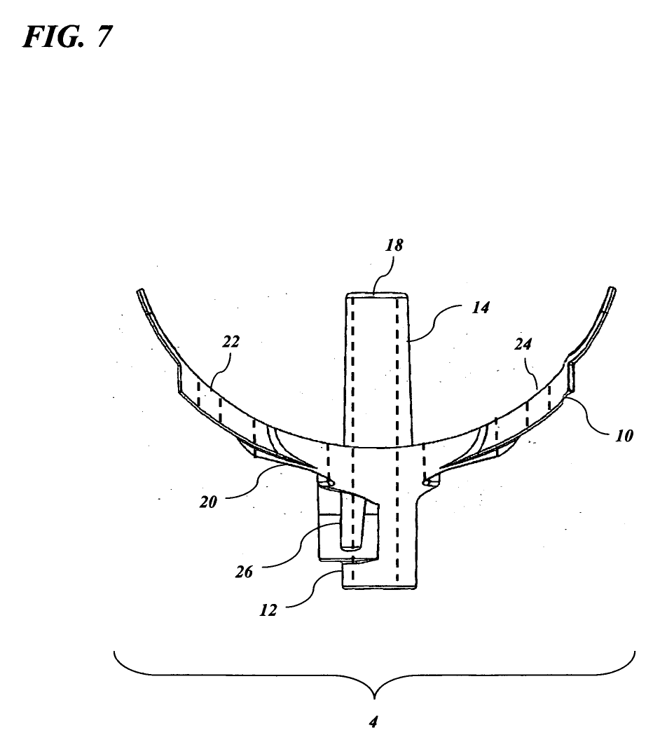

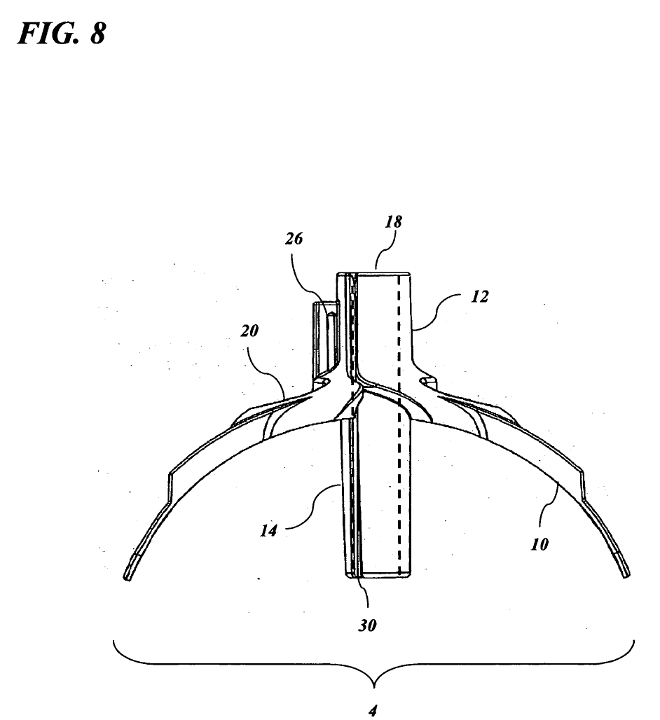

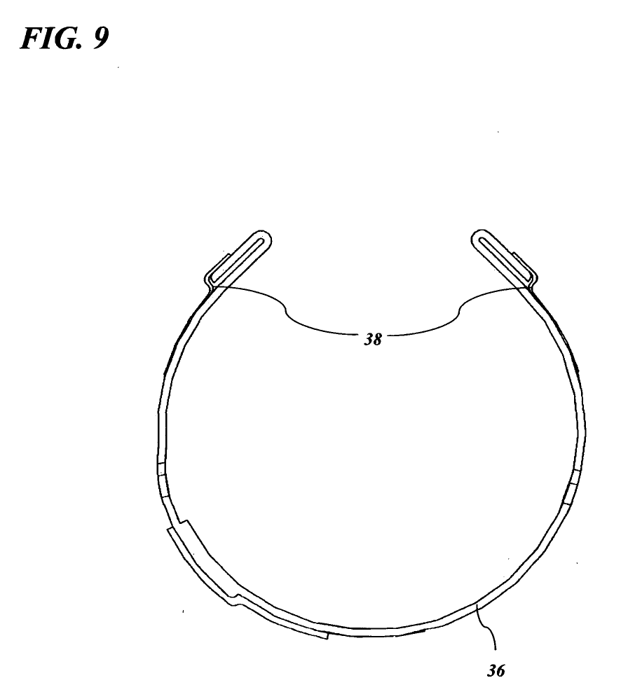

Referring more specifically to the figures, for illustrative purposes the present invention is embodied in the apparatus generally shown in FIG. 1 through FIG. 10.

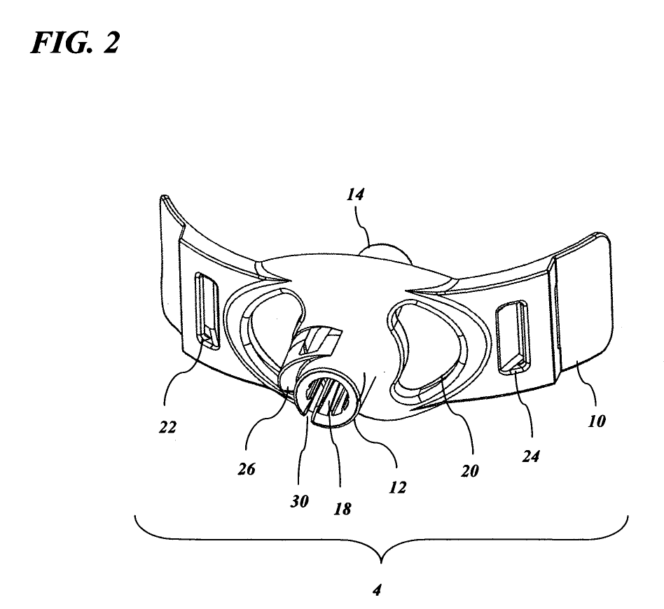

As shown in FIG. 1, the endotracheal tube holder 4 consists of a monolithic flexible structure having integrated therein a base flange 10, an extraoral circumferential guard 12, and an intraoral circumferential guard 14. Base flange 10 has at the outer left and right hand edges cinch plates 22 and 24, respectively, and optionally include one or more access portal 20. Extending from the distal end of extraoral circumferential guard 12, through base flange 10, and to the distal end of intraoral circumferential guard 14 is tube conduit 18. Tube conduit 18 further includes a traverse access way 30 (FIG. 2), which extends the full length of tube conduit 18. Traverse access way 30 is oriented to a side aspect of the endotracheal tube holder 4 such that upon distention of traverse access way 30, an endotracheal tube lumen 40 can be inserted through traverse access way 30 and into tube conduit 18 therein.

As depicted in FIGS. 1 through 8, the endotracheal tube holder 4 includes base flange 10 that extends perpendicular to the access port/circumferential guard elements and is engaged by extensions from a retention band 36 or like device in cinch plates 22 and 24 so as to maintain endotracheal tube holder 4 in an intended position and orientation on the patient. Base flange 10 is an advantageous element of the monolithic design of the endotracheal tube holder 4 as it allows for positioning of the endotracheal tube lumen 40 itself from a stabilized platform without reliance on the integrity of the maxilla-mandibular boney structure for support, a structure that may be severally compromised in accident victims requiring the kind of emergency respiratory care indicative of intubation and endotracheal tube use. To provide a stable platform for the complete endotracheal tube holder 4, base flange 10 has a thickness in the range of 0.10 inch to 0.50 inch, and preferably within the range of 0.15 inch and 0.45 inch inclusively.

Integral to endotracheal tube holder 4 are circumferential guard projections for maintaining optimal flow characteristics of the restrained endotracheal tube, each guard having a length of between 0.25 inch and 6.0 inches, with the range of 0.50 inch to 3 inches being preferred and the range of 1.0 inch to 2.0 inches being most preferred. A first circumferential guard projection, extraoral circumferential guard 12, extends outward from base flange 10 and occupies an extraoral space relative to the patient. Extraoral circumferential guard 12 provides protection against the restrained endotracheal tube lumen 40 from loosing flow characteristics due to external compression or avulsion forces (e.g. kinking of the endotracheal tube 40 on itself or against endotracheal tube holder 4). Of particular concern, endotracheal tube lumen 40 can be become kinked or otherwise impinged due to entrainment with other equipment, movement of the patient during transport, and inadvertent compression by other objects or personnel. The extraoral circumferential guard 12 extends about endotracheal tube lumen 40 as the lumen extends out and away from the patient. The resilient flexible nature of the monolithic construction used throughout endotracheal tube holder 4 increases the resistance of extraoral circumferential guard 12 with restrained endotracheal tube lumen 40 to bend at anything less than an obtuse angle, thus reducing potential for possible restriction of airflow characteristics within endotracheal tube lumen 40. To provide suitable function, extraoral circumferential guard 12 has a thickness in the range of 0.065 to 0.30 inch, preferably within the range of 0.080 inch and 0.25 inch inclusively.

A second circumferential guard projection, intraoral circumferential guard 14, extends from base flange 10 and occupies an intraoral space relative to the patient. Intraoral circumferential guard 14 provides protection against the restrained endotracheal tube lumen 40 from loosing flow characteristics due to compression and abrading action by the patient. Of particular concern, endotracheal tube lumen 40 can be become kinked or otherwise impinged due to involuntary biting by the patient. Further, repeated biting action by a patient can abrade endotracheal tube lumen 40 resulting in possible increased friction contact surfaces on the outside of the lumen that can, in turn, abrade the soft tissue of the patient's perioral and intraoral cavity and if biting is pronounced or prolonged, can ultimately result in ventilation leakage and decreased respiratory support efficiency. The intraoral circumferential guard 14 extends about endotracheal tube lumen 40 as the lumen extends into the trachea of the patient. The resilient flexible nature of the monolithic construction used throughout endotracheal tube holder 4 increases the resistance of intraoral circumferential guard 14 with restrained endotracheal tube lumen 40 to compression and abrasion by the patient without causing undue stress upon the patient's dental structures, thus reducing potential for possible compromise of air flow characteristics. To provide suitable function, intraoral circumferential guard 14 has a thickness of in the range of 0.035 inch to 0.30 inch, preferably within the range of 0.060 inch and 0.25 inch inclusively.

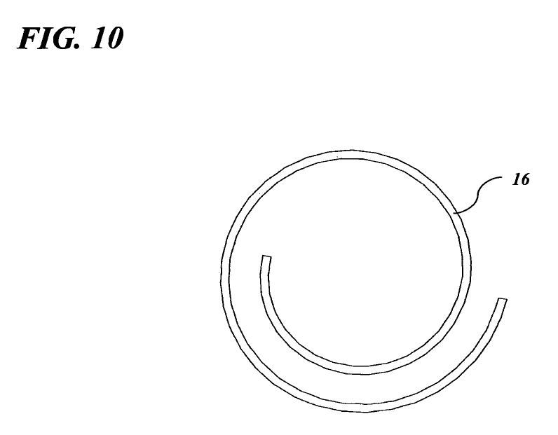

The endotracheal tube holder assembly 4 is adaptable to receiving tubes of diameters ranging from 3.0 mm to 10.5 mm. Endotracheal tubes are inserted through traverse access way 30 and into tube conduit 18. Traverse access way 30 extends the full length of intraoral circumferential guard 14, base flange 10, and extraoral circumferential guard 12. The pathway by which traverse access way 30 extends may be straight, curved, helical, notched and combinations thereof. In a preferred embodiment, traverse access way 30 exhibits at least 10 degrees of rotation from an intraoral space to an extraoral space so that compression of either circumferential guard does not cause tube conduit 18 to open in distention. To prevent inadvertent displacement of an endotracheal tube inserted in tube conduit 18 through traverse access way 30, ring strip 16 (FIGS. 1 and 10) extends around the outer circumference of extraoral circumferential guard 12 and connects back upon itself to prevent traverse access way 30 from opening without direct user manipulation. Ring strip 16 may comprise a mechanical attachment means, a chemical adhesive, and the combinations thereof.

Tube conduit 18 is configured through the total length defined by the monolithic structure of extraoral circumferential guard 12, base flange 10, and intraoral circumferential guard 14. The cross sectional geometry of tube conduit may be straight, angled, curved, or the compound combinations thereof, with a generally elliptical shape being preferred. The cross sectional width or diameter may be constant or may vary along the length of tube conduit 18. Further, it is within the prevue of the present invention that the inner surface of tube conduit 18 may include ribs or other retention modifying geometries in the length or width dimensions, as well as combinations thereof.

The adaptability of the present inventive endotracheal tube holder design allows the device to be used in a broad cross section of patient sizes and ages. Definition of what constitutes an “endotracheal tube” includes but is not limited to one or more airway conduits, an endosurgical lumen, an orogastric tube, an esophageal thermometer, Dobhoff feeding tube and the combinations thereof. Definition of what constitutes a “patient” includes both human and veterinary subjects.

The monolithic nature of the endotracheal tube holder design is further enhanced through incorporation of one or more access portals 20 in base flange 10 for allowing routine patient maintenance. Access portals 20 may have any geometry suitable to the interior bounds described by base flange 10, and may include shapes having rectilinear, elliptic and compound combinations thereof. The geometry of access portal 20 may be completely circumscribed by the material composition of base flange 10, or may have extend to and include an edge profile of said base flange 20. During a course of therapy whereby an endotracheal tube lumen 40 is required, the ability to easily access the intraoral, as well as the pharyngeal and laryngeal regions, is extremely beneficial. It may become necessary to evacuate fluids that accumulate within the regions above the trachea. The incorporation of access portals 20 in base flange 10 allow for medical personnel to readily insert vacuum suction devices to remove accumulated fluids and to apply medicants, particularly when protracted used of an endotracheal tube lumen 40 is required and maintenance of the oral tissues is necessary.

Cinch plates 22 and 24 are provided so as to allow for base flange 10 to be securely positioned in the oral cavity of the patient and yet also provide for periodic repositioning of endotracheal tube holder 4. A cinch plate may include a suitable profile for engaging a strap or other such means employed in retention band 36, including but not limited to posts, buttons, snaps, loops, and crossbar designs. In a preferred embodiment, cinch plates having a crossbar geometry are used to receive a strap that extends under and back over to capture the cross bar element. Retention band 36 may be constructed in part of whole with one or more extensible strap, band or cord elements 38, which themselves may be comprised of woven, nonwoven, foam, or other such suitable materials or combinations thereof. At least one end of band element 38 includes a means for releasably engaging cinch plate 22 or 24 of base flange 10 such that endotracheal tube holder 4 can be applied to the patient with minimal disruption of the patient's head region. Retention band 36 may include additional releasable means, including a means of engaging the other cinch plate and for retention band 36 to engage upon itself at a separation point. Suitable releasable engagement means or fasteners include buckles, snaps, key ways, and hook and loop, with hook and loop fasteners being particularly preferred.

The general construction of functional elements of endotracheal tube holder 4 may comprise any polymer composition suitable for protracted contact with patient dermis and airway ventilation, having a durometer rating of 15 to 110 inclusively as determined by ASTM D2240-00, and preferably a durometer rating of 25 to 100 inclusively. Suitable polymer compositions having a durometer rating within the ranges described include silicon, butyl rubber, SB block polymers, and elastomeric polymers. The polymer composition may further include one or more aesthetic modifying additives or topical treatments (e.g. colorants and pigments) and/or performance modifying additives or topical treatments (e.g. wetting agents). Preferably, endotracheal tube holder 4 is completely radiotransparent so as to not interfere with x-ray or other imaging technologies.

For purposes of manufacturing an endotracheal tube holder 4 in accordance with the present invention, conventional injection molding is suitable wherein a mould having a cavity in the shape of the complete endotracheal tube holder assembly and the complete assembly is cast at one time.

### EXAMPLES

A first device was manufactured in accordance with teachings of the present invention. The device includes the following dimensions:

Base Flange Height: 0.20 inch

Base Flange Width (Left Cinch Plate to Right Cinch Plate): 3.82 inches

Base Flange Inner Diameter (Tube Conduit): 0.28 inch

Extraoral Guard Length: 1.15 inches

Extraoral Guard Outer Diameter: 0.70 inches

Extraoral Guard Inner Diameter (Tube Conduit): 0.40 inch

Intraoral Guard Length: 1.89 inches

Intraoral Guard Outer Diameter: 0.65 inch

Intraoral Guard Inner Diameter (Tube Conduit): 0.40 inch

A second device was manufactured in accordance with teachings of the present invention. The device includes the following dimensions:

Base Flange Height: 0.20 inch

Base Flange Width (Left Cinch Plate to Right Cinch Plate): 2.97 inches

Base Flange Inner Diameter (Tube Conduit): 0.28 inch

Extraoral Guard Length: 1.15 inches

Extraoral Guard Outer Diameter: 0.63 inches

Extraoral Guard Inner Diameter (Tube Conduit): 0.41 inch

Intraoral Guard Length: 1.89 inches

Intraoral Guard Outer Diameter: 0.72 inch

Intraoral Guard Inner Diameter (Tube Conduit): 0.44 inch

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art. In the appended claims, reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the disclosure and present claims. Moreover, it is not necessary for a device or method to address each and every problem sought to be solved by the present invention, for it to be encompassed by the disclosure and present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. An endotracheal tube holder comprised of:
a. a base flange;
b. an extraoral circumferential guard extending from said base flange;
c. an intraoral circumferential guard extending from said base flange;
d. an endotracheal tube conduit described interior to said endotracheal tube holder, extending through each of said base flange, extraoral circumferential guard and intraoral circumferential guard;
wherein said base flange, extraoral circumferential guard and intraoral circumferential guard are integral to and monolithically formed into one another.

2. An endotracheal tube holder as in claim 1, wherein said extraoral circumferential guard further comprises a ring strip.

3. An endotracheal tube holder as in claim 1, wherein said base flange is further comprised of at least one access portal.

4. An endotracheal tube holder as in claim 1, wherein said endotracheal tube holder is composed of a polymeric composition having a durometer rating of equal to or greater than 15 and less than or equal to 110.

5. An endotracheal tube holder as in claim 4, wherein said polymeric composition is silicone.

6. An endotracheal tube holder comprised of:
a. a base flange having two opposed cinch plates;
b. an extraoral circumferential guard extending from said base flange;
c. an intraoral circumferential guard extending from said base flange;
d. an endotracheal tube conduit described interior to said endotracheal tube holder, extending through each of said base flange, extraoral circumferential guard and intraoral circumferential guard;
wherein said base flange, extraoral circumferential guard and intraoral circumferential guard are integral to and monolithically formed into one another.

7. An endotracheal tube holder as in claim 1, wherein said extraoral circumferential guard further comprises a ring strip.

8. An endotracheal tube holder as in claim 1, wherein said base flange is further comprised of at least one access portal.

9. An endotracheal tube holder as in claim 1, wherein said endotracheal tube holder is composed of a polymeric composition having a durometer rating of equal to or greater than 15 and less than or equal to 110.

10. An endotracheal tube holder as in claim 9, wherein said polymeric composition is silicone.

11. An endotracheal tube holder as in claim 1, wherein said endotracheal tube holder further comprises a retention band engaged upon said cinch plates.

12. An endotracheal tube holder as in claim 11, wherein said retention band comprises one or more extensible cord elements.

13. A method of using an endotracheal tube holder comprised of
a. inserting an endotracheal tube into a patient through an intubation procedure;
b. obtaining an endotracheal tube holder comprised of:
i. a base flange having two opposed cinch plates and a retention band associated therewith;
ii. an extraoral circumferential guard extending from said base flange;
iii. an intraoral circumferential guard extending from said base flange;
iv. an endotracheal tube conduit described interior to said endotracheal tube holder, extending through each of said base flange, extraoral circumferential guard and intraoral circumferential guard;
wherein said base flange, extraoral circumferential guard and intraoral circumferential guard are integral to and monolithically formed into one another;
c. engaging said retention band to said intubated patient; and
d. inserting said endotracheal tube into said endotracheal tube conduit of said endotracheal tube holder.

14. A method as described in claim 13, wherein said extraoral circumferential guard further comprises a ring strip.

15. A method as described in claim 14, wherein said inserted endotracheal tube conduit is closed through action of said ring strip.
